(19) United States
(12) Patent Application Publication
(10) Pub. No.: US 2020/0113743 A1
(43) Pub. Date: Apr. 16, 2020

(54) TEXTILE ORTHOSIS HAVING A TAB

(71) Applicant: LABORATOIRES INNOTHERA, Arcueil (FR)

(72) Inventor: Jean-Pierre SLASKI, Brantigny (FR)

(73) Assignee: LABORATOIRES INNOTHERA, Arcueil (FR)

(21) Appl. No.: 16/599,676

(22) Filed: Oct. 11, 2019

(30) Foreign Application Priority Data
Oct. 12, 2018 (FR) 18 59460

(51) Int. Cl. A61F 13/08; A61F 13/00

(52) U.S. Cl. CPC A61F 13/08; A61F 13/00021

(57) ABSTRACT

An orthosis chosen from an elastic venous retention orthosis and an elastic venous compression orthosis is intended to treat a venous disorder of a lower limb of a patient in a service position in which it is fitted on the lower limb. The orthosis has a leg part, a ribbed top configured to maintain the orthosis in the service position, and a tab fixed on the ribbed top in such a way as to be accessible to the patient in the service position.

### TECHNICAL FIELD

The present invention relates to an elastic venous compression or retention orthosis (or EVC orthosis, or “orthosis”) which, in the service position, extends substantially as far as the knee of the lower limb of a patient on which it is fitted.

### PRIOR ART

An EVC orthosis of this kind, formerly known by the term “retention stocking (or hose)” or “retention tights”, is a textile medical device producing a therapeutic effect through retention and/or compression of the lower limbs, as opposed to “support stockings” (or even “support hose” or “anti-fatigue stockings”) and “fashion stockings”, which are not medical devices with a therapeutic purpose.

The pressure profile exerted by an orthosis usually decreases upward from the ankle.

Depending on the type of orthosis, the pressure measured at the ankle can in particular vary from 10 to over 36 mmHg (i.e. 13 to 48 hPa, although mmHg is commonly used as the unit of measurement of pressure in the field of phlebology and of medical compression).

The orthoses are divided into four textile classes according to the ASQUAL system, from class I (13 to 20 hPa≈10 to 15 mmHg at the ankle) to class IV (>48 hPa≈>36 mmHg at the ankle).

The upper part of the orthosis, which defines the opening via which it is fitted, traditionally has a ribbed top. A ribbed top is a band of knitting which is extensible in the circumferential direction and which, once the orthosis has been fitted on the limb, has the function of locally exerting a textile pressure for maintaining the orthosis in position.

However, the elasticity of the ribbed top, necessary for maintaining the orthosis in the service position, makes the orthosis difficult to put on.

An aim of the invention is to make available an orthosis that at least partially overcomes this problem.

### SUMMARY OF THE INVENTION

The invention proposes an orthosis chosen from an elastic venous retention orthosis and an elastic venous compression orthosis, said orthosis being intended to treat a venous disorder of a lower limb of a patient in a service position in which it is fitted on said lower limb via an opening, said orthosis having a leg part and a ribbed top, preferably of constant height, configured to maintain the orthosis in the service position, said orthosis extending, in the service position, substantially as far as the knee of said lower limb.

According to the invention, the orthosis is notable in that it has a tab fixed to the ribbed top in such a way as to be accessible to the patient in the service position.

Preferably, the tab is arranged in the rear part of the ribbed top in such a way as to be in contact with, and preferably to bear on, the rear part of the patient's calf in the service position.

The patient can pull the tab when putting on the orthosis. This advantageously makes the orthosis easier to put on.

An orthosis according to the invention can also have one or more of the following optional and preferred features:
the tab extends in the continuation of the ribbed top, starting from the upper edge of the ribbed top, so as to face the patient's calf in the service position;
the tab extends on an outer face of the ribbed top;
the tab is knitted in continuity with the ribbed top, making its production easier;
the tab is an added-on piece which preferably has the shape of a tongue, a ring or a rod;
the added-on piece is made of woven fabric or knitted fabric or made of plastic;
the tab has a height greater than 0.5 cm and less than 3 cm, and/or a maximum length greater than 1 cm and less than 8 cm, making it easier to grip;
the tab is configured in such a way as to bear elastically on said lower limb in said service position, which advantageously limits the interactions of the tab with its surroundings, for example with trousers;
the tab has an appearance differing from the ribbed top, which advantageously makes the tab easier to see;
the orthosis has just one tab.

### BRIEF DESCRIPTION OF THE FIGURES

Further features and advantages of the invention will become clear from reading the following detailed description and from studying the attached drawing in which:

FIG. 1 shows a schematic side view of an orthosis according to the invention;

FIG. 2 shows a plan view of the orthosis from FIG. 1.

The figures depict the median transverse plane P_{M} passing along the main direction X of the orthosis and separating the space, and in particular the ribbed top, into a front part P_{AV} and a rear part P_{AR}. A “transverse” plane is a plane parallel to the general plane of the patient's body in the service position.

The figures also depict the median longitudinal plane P_{L} passing along the main direction X of the orthosis and separating the space, and in particular the ribbed top, into a right part P_{DR} and a left part P_{GA}. A “longitudinal” plane is a plane that contains the main direction X and that is perpendicular to the general plane of the patient's body in the service position.

In the figures, the same references are used to designate identical or functionally equivalent elements.

### DEFINITIONS

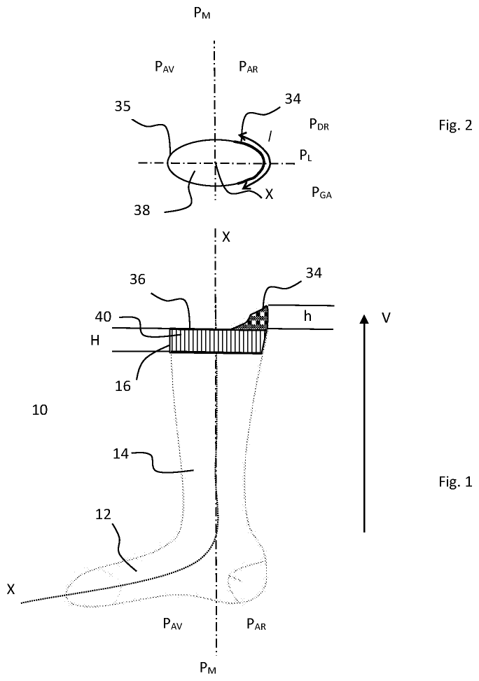

The “service position” of an orthosis corresponds to a position of use, that is to say a position in which it is fitted on a lower limb of a patient who is standing up.
The “at rest” or “flat” position of an orthosis is a position in which the orthosis is not being worn, for example before the first use, and extends, seen from the side, in the same way as in the service position, as in FIG. 1.
The pressure exerted at a point by an orthosis is, according to Laplace's law, a pressure referred to as a “textile pressure” within the meaning of French standard NF G30-102b, inversely proportional to the radius of curvature at this point.
The “altitude” corresponds to a level in the vertical direction V (shown in FIG. 1) in the service position. A “height” is a difference in altitude.
A “stitch” is a way of interlacing the knit and weft threads.

Unless indicated to the contrary, the dimensions, such as the height, and the positions, such as “front”, “rear”, “upper” or “lower”, are given with reference to the at rest position.

Unless indicated to the contrary, “having”, “comprising”, “including”, or variations thereof, correspond to non-exclusive inclusion.

### DETAILED DESCRIPTION

Reference sign 10 designates in a general manner an orthosis according to the invention, extending along the main direction X.

The orthosis 10, of overall anatomical shape, comprises
a foot part 12 which envelops the foot, extending from the toes as far as the malleoli, covering the instep,
a leg part 14 which is extensible in a longitudinal direction (that is to say in the main direction X) and in the circumferential direction (radial extensibility), extending from the malleolar region, enveloping the ankle and the calf, up to a level situated below the knee; and
a ribbed top 16 extensible mainly in the circumferential direction, typically a knitted part.

An orthosis according to the invention, as shown in FIG. 1, takes the form of a knee-high sock, referred to as “knee-length” or “half-stocking” in the “AD” format, meaning that the ribbed top reaches to just below the knee when the orthosis is in the service position. Outwardly, it therefore has the same appearance as traditional “fashion” socks, but differs from these in terms of the choice of thread and a knit that allows effective therapeutic compression to be obtained, preferably compression of class II.

The orthosis 10 can be without a foot part 12 (stocking or hose of the “open foot” type).

The orthosis 10 is conventionally made from a knitted stitch and, in the leg part, incorporates an elastic weft thread, generally made of a covered elastane.

The nature of the threads and the stitches used are adapted depending on the action and the appearance that are desired for the different parts of the orthosis. This adaptation does not pose any particular difficulty to a person skilled in the art.

The ribbed top 16 continues the leg part 14 and, in the service position, forms a substantially cylindrical belt, preferably of constant height H.

The opening 38 of the orthosis is delimited by a rim 35, which is defined by the ribbed top and the tab and which extends in the continuation of the ribbed top.

The height H of the ribbed top 16 is preferably more than 2.0 cm, preferably more than 3.0 cm, preferably more than 4.0 cm, and/or preferably less than 8 cm, preferably less than 6 cm, preferably less than 5 cm.

The outer face of the ribbed top 16 preferably has ribs 40.

Preferably, the ribbed top 16 is knitted in continuity with the leg part 14. In one embodiment, the only difference between the leg part and the ribbed top of the ribbed top lies in the stitch.

The tab 34 protrudes upward from the upper edge 36 of the ribbed top 16.

The maximum height h of the tab 34, measured from the upper edge 36, is preferably more than 0.5 cm, preferably more than 0.8 cm, preferably more than 1 cm, and/or preferably less than 3 cm, preferably less than 2 cm.

The height h is preferably substantially constant along the entire length of the tab 34.

The maximum length/of the tab 34 is preferably more than 1 cm, preferably more than 2 cm, preferably more than 3 cm, and/or preferably less than 8 cm, preferably less than 5 cm, preferably less than 4 cm.

The maximum thickness of the tab 34 is preferably more than 0.5 mm and/or preferably less than 5 mm, preferably less than 3 mm, preferably less than 2 mm.

Preferably, the thickness of the tab 34 is substantially constant. Preferably, the thickness of the tab 34 is substantially equal to the thickness of the ribbed top. Preferably, the border between the tab 34 and the ribbed top 16 has no discontinuity, such that this border is smooth to the touch.

The structure, in particular the stitch, of the tab 34 can be identical to or different than that of the ribbed top 16.

The tab 34 can be knitted and have a network of stitches that are produced by means of a stitch thread and a weft thread interlaced between said stitches.

The stitch thread of the tab can be identical to or different than the stitch thread of the ribbed top 16.

The weft thread of the tab can be identical to or different than the weft thread of the ribbed top 16.

Preferably, the weft thread of the tab 34 is a covered thread, preferably a double-covered thread.

Preferably, the linear density of the core of the weft thread of the tab 34 is below 150 dTex, preferably below 140 dTex, preferably below 135 dTex, and/or above 100 dTex, preferably above 110 dTex, preferably above 120 dTex, preferably above 125 dTex.

Preferably, the linear density of the covering thread of the weft thread of the tab 34 is below 40 dTex, preferably below 30 dTex, preferably below 25 dTex, and/or above 10 dTex, preferably above 15 dTex, preferably above 20 dTex.

Preferably, the tab 34 is knitted in continuity with the ribbed top 16, preferably by augmentation or diminution.

Preferably, the tab 34 is preferably configured in such a way as to bear elastically on the lower limb of the patient in the service position.

The tab 34 can also be added on, preferably sewn onto the ribbed top 16. However, the tab 34 is preferably fixed such that it protrudes from the upper edge 36 of the ribbed top 16 at rest and in the service position, unlike a label in particular.

The tab is preferably knitted, but it can also be of any other material compatible with contact with the skin, for example a biocompatible polymer material.

In one embodiment, the tab 34 has the same color, preferably the same appearance, as the ribbed top 16. The border between the ribbed top 16 and the tab 34 can be invisible. This makes the tab more discreet.

In one embodiment, the tab 34 has an appearance differing from the ribbed top 16 and making it more visible. This can improve the originality of the orthosis.

In one embodiment, the tab 34 carries information, for example concerning the brand and/or the size and/or the date of production of the orthosis, and/or concerning the manufacturer and/or distributor of the orthosis.

The particular appearance of the tab 34 can thus serve as a unique identifier for all the orthoses of a range of orthoses, or for a specific size, irrespective of the particular orthosis range of a manufacturer, or for all the orthoses of a manufacturer.

In one embodiment, the tab 34 carries a microchip.

The information and/or the microchip are/is preferably carried on the inner face of the tab 34, that is to say on that part of the tab which, in the service position, is oriented toward the patient's skin or is even in contact with the patient's skin. Preferably, the information and/or the microchip are/is carried exclusively on the inner face of the tab 34.

Preferably, the orthosis has fewer than four, preferably fewer than three, tabs 34, preferably a single tab 34.

Preferably, a tab 34 is arranged at least partially, preferably completely, on the rear part P_{AR} of the ribbed top.

Preferably, a tab 34 is arranged in such a way as to bear, in the service position, on the patient's calf, preferably on the rear part of the patient's calf, preferably elastically.

Discreetness and comfort are improved in this way.

Preferably, a tab 34 is arranged in such a way as to be centered on the median longitudinal plane P_{L}.

Preferably, a tab 34 is arranged in the rear part of the orthosis, remote from the foot part 12.

The position, structure and size of the tab 34 make it easy for the patient to grip. The patient can thus easily widen the opening of the orthosis when putting the latter on. He can also more easily grasp the upper part of the orthosis in order to take the latter off.

Of course, the invention is not limited to the embodiments described and shown, which have been provided only for illustrative purposes.

In particular, the tab can have shapes other than the tongue shape shown, although the latter constitutes a preferred embodiment.

The tab can also be fixed on the outer face of the ribbed top, that is to say on the face opposite the face in contact with the patent's skin in the service position.

1. Orthosis chosen from an elastic venous retention orthosis and an elastic venous compression orthosis, said orthosis being intended to treat a venous disorder of a lower limb of a patient in a service position in which it is fitted on said lower limb, said orthosis extending, in the service position, substantially as far as the knee of said lower limb, and having:
a leg part,
a ribbed top configured to maintain the orthosis in the service position, and
a tab fixed on the ribbed top in such a way as to be accessible to the patient in the service position.

2. Orthosis according to claim 1, in which the tab is arranged in the rear part of the ribbed top, in such a way as to be in contact, in the service position, with the rear part of the patient's calf.

3. Orthosis according to claim 2, in which the tab is configured in such a way as to bear elastically on the lower limb in the service position.

4. Orthosis according to claim 1, in which said tab has a height greater than 0.5 cm and less than 3 cm, and/or a length greater than 1 cm and less than 8 cm.

5. Orthosis according to claim 1, in which said tab is knitted in continuity with the ribbed top or is an added-on piece.

6. Orthosis according to claim 5, in which the added-on piece has the shape of a tongue, a ring or a rod.

7. Orthosis according to claim 1, in which said tab has an appearance differing from the ribbed top.

8. Orthosis according to claim 1, in which the tab is an added-on piece having the general shape of a ring or of a rod.

9. Orthosis according to claim 1, of class I, II, III or IV according to the ASQUAL system.
